(19) United States
(12) Patent Application Publication
(10) Pub. No.: US 2006/0264943 A1
(43) Pub. Date: Nov. 23, 2006

(54) Intramedullary nail

(76) Inventor: Poonung Chieng, Shanghai (CN)

Correspondence Address: BACON & THOMAS, PLLC, 625 SLATERS LANE, FOURTH FLOOR, ALEXANDRIA, VA, 22314, US

(21) Appl. No.: 10/549,256

(22) PCT Filed: Mar. 15, 2004
(86) PCT No.: PCT/CN04/00205

§ 371 Date: Sep. 13, 2005

(30) Foreign Application Priority Data
Mar. 18, 2003 (EP) 03100693.5

(51) Int. Cl. A61F 2/30

(52) U.S. Cl. 606/62

(57) ABSTRACT

The present invention relates to an intramedullary nail. The said intramedullary nail comprises a hollow shank which is longitudinally slotted at its front end. The said intramedullary nail also comprises a mobilizable bar which passes through the said hollow shank. An expansion head is provided at the front end of the said mobilizable bar. A nut can be screwed onto the rear end of the said mobilizable bar.

### BACKGROUND OF THE INVENTION

1. Field of the Invention

The present invention relates to an intramedullary nail, and more particularly to a device that can be passed directly through a broken bone to secure the device at the position of the broken bone.

2. Description of the Related Art

Referring to FIG. 1 for the method of connecting a broken bone 1, a nail 2 is passed through a medullary cavity. Since the nail 2 is in a straight and flat shape, therefore it is necessary to have an accessory nail 3 nailed into the radial direction of the surface of the medullary cavity to prevent the nail 2 from being fallen off from the medullary cavity when the nail 2 enters into the medullary cavity. In the meantime, it is necessary to use X-ray to complete the process of precisely pressing the plurality of accessory nails 3 on the nail 2 and connecting a broken bone by a connecting means during a surgery in order to precisely connect a broken bone. Such requirement not only increases the operation time, but also make it difficult for the newly grown blood vessels and bone scars to effectively cover the bone and fuse the broken bone. This shortcoming makes the application impractical and the patient painful, and it is thus a major issue to medical people and patients.

To overcome the foregoing shortcoming, a crotched intramedullary nail was introduced. Although the crotched intramedullary nail of this sort has a crotched member disposed at the front end and pressing against the medullary cavity to directly fix the intramedullary nail into the medullary cavity when the crotched intramedullary nail enters into the medullary cavity, yet the crotched angle at the front end of the intramedullary nail is too small, and thus the intramedullary nail cannot press against the medullary cavity. If the crotched angle at the front end of the intramedullary nail is too large, then the intramedullary nail cannot enter into the medullary cavity easily. In that case, a surgery tong is required to clamp the intramedullary nail. Since the surface of the intramedullary nail is very smooth, the tong cannot clamp the intramedullary nail easily and thus making the application very inconvenient.

### SUMMARY OF THE INVENTION

Therefore, it is a primary objective of the present invention to overcoming the shortcomings of the prior art by providing an intramedullary nail that can be directly passed through a broken bone to connect the broken bone.

The intramedullary nail comprises a hollow shank, a crotched member extended axially and disposed at an end of the hollow shank, a plurality of grooves extended axially between the crotched members, a mobilizable bar passed through the hollow shank, and an expansion head disposed at an end that passes through the hollow shank, such that the crotched member of the hollow shank can enter from an end of a broken bone and passed through the broken position. A fixing means is pushed towards the hollow shank when the crotched member is pushed to the other end, so that the mobilizable bar in the hollow shank will move towards the hollow shank as the fixing means moves, and the expansion head originally situated outside the crotched member is moved into the hollow shank accordingly. As a result, the plurality of crotched members is pressed by the expansion head and extended slowly to a predetermined angle to fix the hollow shank and thus achieving the purpose of connecting a broken bone.

The mobilizable bar further comprises a fixing means disposed at an end away from the crotched member, so that when the hollow shank is pushed into the broken medullary cavity, the mobilizable bar in the hollow shank can be fixed into its position by the blocking of the fixing means, and thus preventing the plurality of crotched members from being pressed by the expansion head or opening when the crotched member has not been pushed to a predetermined position.

### BRIEF DESCRIPTION OF THE DRAWINGS

FIG. 1 is a schematic view of a prior art device;

FIG. 2 is a perspective view of the present invention;

FIG. 3 is a schematic view of the movements according to the present invention;

FIG. 4 is a schematic view of an intramedullary nail entering into the medullary cavity according to the present invention; and

FIG. 5 is a schematic view of fixing an intramedullary nail into the medullary cavity according to the present invention.

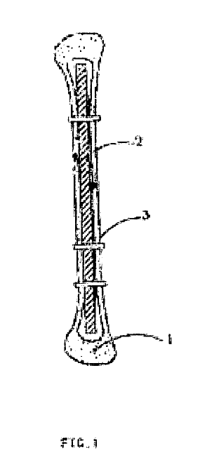

### DETAILED DESCRIPTION OF THE PREFERRED EMBODIMENTS

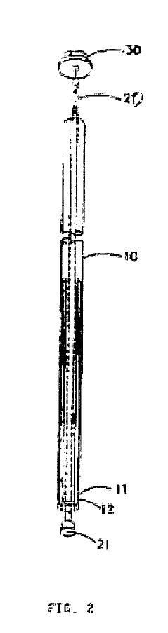

Referring to FIG. 2 for the intramedullary nail of the present invention, the intramedullary nail is a device capable of connecting a broken medullary cavity, and the device comprises a hollow shank 10, which is a hollow member in this preferred embodiment, and the hollow shank 10 includes more than one crotched member 11 extended axially from the periphery at an end of the hollow shank 10, and a groove 12 extended axially between the plurality of crotched members 11, so that the crotched member 11 can be opened. The hollow shank 10 includes a mobilizable bar 20 which is a rod passing through the hollow shank 10 in this preferred embodiment, and the mobilizable bar 20 includes an expansion head 21 disposed at an end where the mobilizable bar 20 passes through the crotched member 11, and the expansion head 21 is a conical member in this preferred embodiment that allows the expansion head 21 to enter into the hollow shank 10 easily. Further, the mobilizable bar 20 includes a fixing means 30 exposed from another end of the hollow shank 10, and the fixing means 30 is a nut in this preferred embodiment. The mobilizable bar 20 further includes a latching section 22 corresponding to the fixing means 30, and the latching section 22 is a thread in this preferred embodiment.

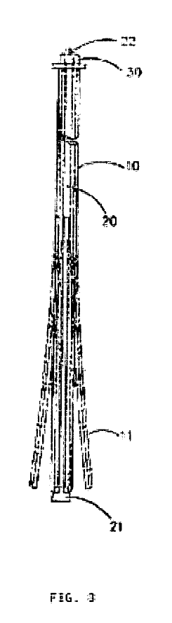

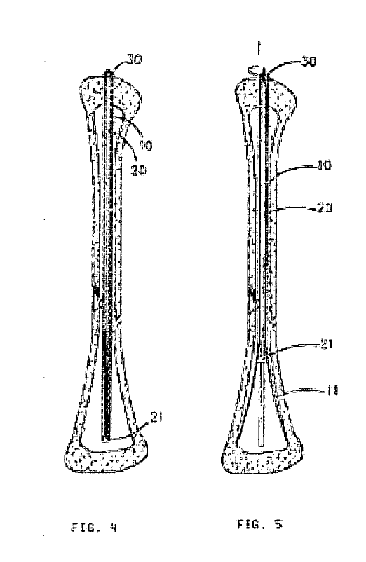

Referring to FIGS. 3 to 5 for the use of the present invention, the crotched member 11 of the hollow shank 10 enters into a medullary cavity from an end of the medullary cavity, and the mobilizable bar 20 of the hollow shank 10 can be fixed into its position by the blocking of the fixing means 30 when the hollow shank 10 is pushed into the broken medullary cavity, so that the expansion head 21 outside the crotched member 11 will not slide near to each crotched member 11, and the crotched member 11 will not open when it is not pushed to a predetermined position. The hollow shank 10 is passed through the broken position from an end of the broken medullary cavity, and finally the fixing means 30 moves towards the hollow shank 10 when the hollow shank 10 enters into another end of the broken medullary cavity, so that the mobilizable bar 20 in the hollow shank 10 can move in the hollow shank 10 according to the movement of the fixing means 30. The expansion head 21 originally situated on the crotched member 11 moves into each crotched member 11 accordingly, so that the crotched members 11 are pressed by the expansion head 21 and slowly extended into a predetermined angle to fix the hollow shank 10 and achieve the purpose of connecting the broken medullary cavity.

What is claimed is:

1. An intramedullary nail, comprising:
a hollow shank, having a plurality of crotched members disposed along the axial direction and extended from an end of said hollow shank; and
a mobilizable bar, passing through said hollow shank and having an expansion head disposed at an end where said mobilizable bar passes through said crotched member, and said mobilizable bar being expanded from an end proximate to said crotched member to another end and said mobilizable bar having a fixing means exposed from another end of said hollow shank.

2. The intramedullary nail of claim 1, wherein said expansion head is increasingly expanded from an end proximate to said hollow shank to another end.

3. The intramedullary nail of claim 1, wherein said fixing means is a nut.
